(19) United States
(12) Patent Application Publication
(10) Pub. No.: US 2010/0196222 A1
(43) Pub. Date: Aug. 5, 2010

(54) AIR CLEANING APPARATUS

(75) Inventors: Takuji Kosugi, Kanagawa (JP); Nobuhiro Nishita, Kanagawa (JP)

Correspondence Address: SUGHRUE-265550, 2100 PENNSYLVANIA AVE. NW, WASHINGTON, DC, 20037-3213, US

(73) Assignee: FUJIFILM CORPORATION, Tokyo (JP)

(21) Appl. No.: 12/678,556

(22) PCT Filed: Sep. 19, 2008
(86) PCT No.: PCT/JP2008/067586

§ 371 Date: Mar. 17, 2010

(30) Foreign Application Priority Data
Sep. 20, 2007 (JP) 2007-243516
Dec. 27, 2007 (JP) 2007-337205

(51) Int. Cl. A61L 9/12

(52) U.S. Cl. 422/186

(57) ABSTRACT

An air cleaning apparatus, includes: a casing main body that includes: an air intake portion; and an air exhaust portion; an air blast portion that sends air to a flow path; a photocatalyst filter that has a layer including a photocatalyst; a light-emitting portion that irradiates the photocatalyst filter with light; and an antibody filter that includes a harmful substance removal material constituted by supporting an antibody on a carrier, wherein: a first light-shielding member that allows the air to flow and shields transit of the light in a state seen from the air flow direction is provided between the light-emitting portion and the antibody filter; and the first light-shielding member includes: at least one frame body; and a plurality of light-shielding plates formed on the at least one frame body and arrayed in such a state as being inclined at the same angle respectively.

### TECHNICAL FIELD

The present invention relates to an air cleaning apparatus that decomposes organic materials using a photocatalyst and selectively deactivates bacteria, viruses and the like with an antibody filter, for the purpose of odor neutralization, deodorization, sterile filtration and the like.

Conventionally, regarding photocatalyst filters for the purpose of deactivation of viruses and air cleaning apparatuses provided with such photocatalyst filter, for example, there are ones as shown in JP-A-2005-342142. The air cleaning apparatus in JP-A-2005-342142 is an air cleaning apparatus that is provided with virus removal capability of deactivating and annihilating viruses through immune antibody reaction, and that, in addition, maintains the effect on deactivating viruses of various types with an electrostatic filter or photocatalyst filter.

### BACKGROUND ART

Incidentally, for a light source for irradiating a photocatalyst filter with light, generally UV rays are employed. But, when UV rays are irradiated to an antibody filter, antibodies included in the antibody filter are destroyed to lower the effect of catching viruses and bacteria.

When producing an antibody, for example, from the serum of an animal, the price of the antibody is so high as seven million yens per 1 kg in the state of being dissolved in the serum. In accordance with purposes, further purification or pulverization is performed, to raise the price further. Accordingly, in the case where an antibody is destroyed, there arises the necessity to support an excess amount corresponding to the amount to be destroyed, which constitutes the cause of a serious cost increase.

Above-described conventional air cleaning apparatuses have such constitution that an antibody filter is arranged on the uppermost stream side or the lowermost stream side in the flow path of air. When it is arranged on the lowermost stream side, it is expected that the antibody filter is irradiated by UV rays from a light source portion to reduce the filter effect of the antibody filter. On the other hand, if a shielding plate or the like is provided between the antibody filter and the light source portion while taking the effect of UV rays on the antibody filter into consideration, it is feared that the flow of air is blocked to decrease the air volume.

The invention was accomplished with the view of the above circumstances, and the purpose thereof is to provide an air cleaning apparatus capable of preventing the reduction of the filter effect caused by the irradiation of UV rays to an antibody filter and capable of preventing the lowering of the air volume.

### DISCLOSURE OF THE INVENTION

The aforementioned purpose of the present invention can be achieved by following constitutions.

(1) An air cleaning apparatus for decomposing an organic material using a photocatalyst, the apparatus comprising:

a casing main body that includes:
an air intake portion that takes air in an inside of the casing main body; and
an air exhaust portion that sends air away to an outside of the casing main body;

an air blast portion that sends air to a flow path formed between the air intake portion and the air exhaust portion;

a photocatalyst filter that has a layer including a photocatalyst and is arranged in the flow path;

a light-emitting portion that irradiates the photocatalyst filter with light; and

an antibody filter that includes a harmful substance removal material constituted by supporting an antibody on a carrier and is arranged in the flow path,

wherein:

a first light-shielding member that allows the air to flow and shields transit of the light in a state seen from the air flow direction is provided between the light-emitting portion and the antibody filter; and

the first light-shielding member includes:

at least one frame body that is arranged in the flow path; and

a plurality of light-shielding plates that are formed on the at least one frame body and arrayed in such a state as being inclined at the same angle respectively.

(2) The air cleaning apparatus as described in (1) above,

wherein at least either one of an antibacterial agent and an antifungal agent is supported on the antibody filter.

(3) The air cleaning apparatus as described in (1) or (2) above,

wherein the antibacterial agent and the antifungal agent is an organic acid silver salt.

(4) The air cleaning apparatus as described in (3) above,

wherein the organic acid silver salt has from 14 to 24 carbon atoms and is linear.

(5) The air cleaning apparatus as described in any one of (1) to (4) above,

wherein the first light-shielding member includes a plurality of frame bodies each having the plurality of light-shielding plates, the plurality of frame bodies being disposed in a superimposed state, and

adjacent two frame bodies are arranged so as to have respective inclination directions of the plurality of light-shielding plates inverse to each other.

(6) The air cleaning apparatus as described in any one of (1) to (5) above,

wherein each of the plurality of light-shielding plates is inclined in a range of from 30 degrees to 50 degrees relative to the horizontal direction.

(7) The air cleaning apparatus as described in any one of (1) to (6) above, further comprising:

a second light-shielding member arranged near a lower stream side of the air intake portion in the flow path, the second light-shielding member being the same as the first light-shielding member.

The air cleaning apparatus according to the invention permits the air flow and, at the same time, prevents the irradiation of the antibody filter by the light from the light-emitting portion, by plural light-shielding plates provided for the light-shielding member. This prevents the breakdown of the antibody on the antibody filter by the effect of UV rays in the light, to make it possible to prevent the lowering of the filter effect of the antibody filter. In addition, the light-shielding member allows the air to flow through the interspaces between light-shielding plates not to hinder the air flow in the flow path, and thus the lowering of the air volume can be prevented.

### BRIEF DESCRIPTION OF THE DRAWING

FIG. 1 is a drawing showing the constitution of one exemplary embodiment of the air cleaning apparatus according to an aspect of the present invention;

FIG. 2 is drawing showing the air cleaning apparatus in FIG. 1 seen from the air intake side;

FIG. 3 is a drawing showing the air cleaning apparatus in FIG. 1 seen from the air exhaust side;

FIG. 4 is a cross-sectional view obtained by cutting the air cleaning apparatus in FIG. 1 along a cross-section parallel to the air flow path;

FIG. 5 is a block diagram showing the control system of the air cleaning apparatus according to the exemplary embodiment;

FIG. 6 is a perspective view showing the constitution of the light-shielding member;

FIG. 7 is a cross-sectional view seen from the direction of A-A line in FIG. 6; and

FIG. 8 is a partial cross-sectional view showing a modified exemplary example of the light-shielding member.

### BEST MODE FOR CARRYING OUT THE INVENTION

Hereinafter, exemplary embodiments of the present invention will be described in detail on the basis of the drawings.

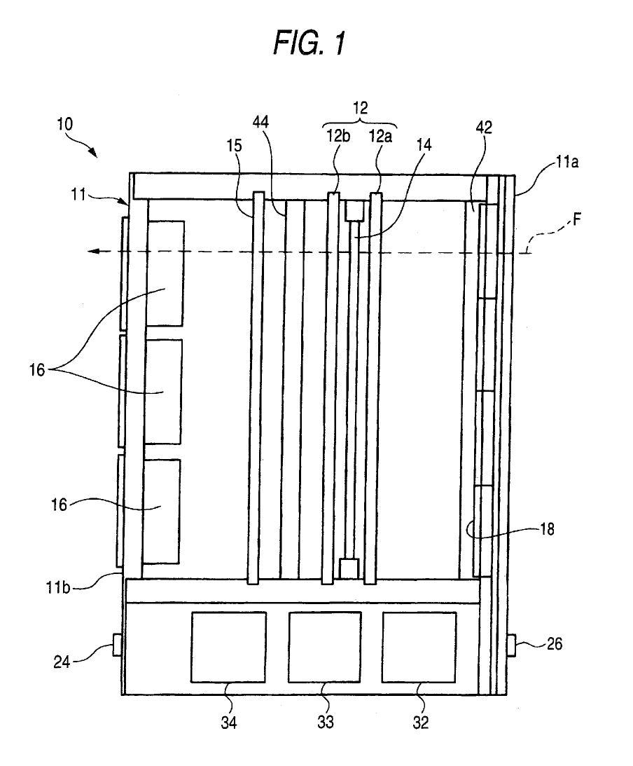

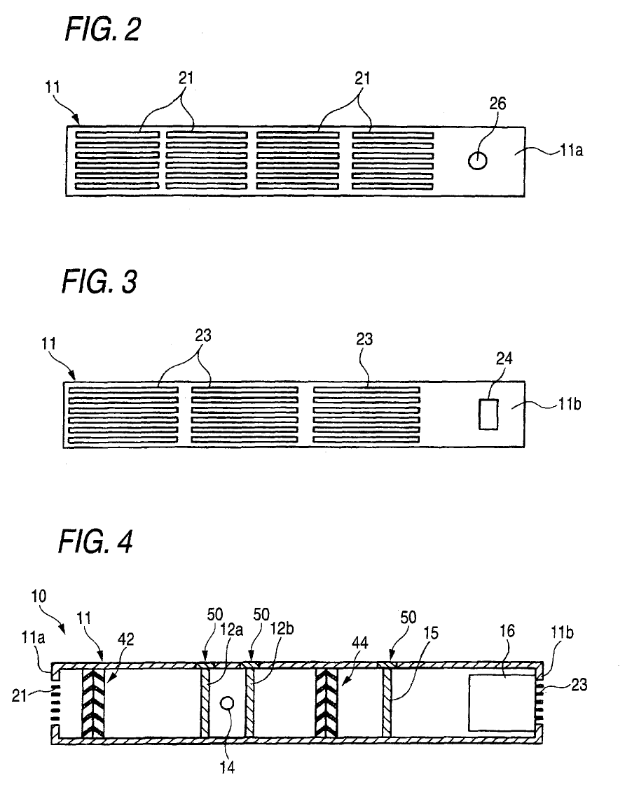

FIG. 1 is a drawing showing the constitution of one exemplary embodiment of the air cleaning apparatus according to an aspect of the invention. FIG. 2 is a drawing showing the air cleaning apparatus in FIG. 1 seen from the air intake side. FIG. 3 is a drawing showing the air cleaning apparatus in FIG. 1 seen from the air exhaust side. FIG. 4 is a cross-sectional view obtained by cutting the air cleaning apparatus in FIG. 1 along the cross-section parallel to the air flow path.

The air cleaning apparatus 10 is provided with a casing main body 11 having a prescribed space therein and an approximately rectangular shape. As shown in FIG. 2, there are formed plural air intake openings 21 in the side face 11a on the air intake side of the casing main body 11. These air intake openings 21 function as air intake portions for taking air into the inside of the casing main body 11. Further, as shown in FIG. 3, there are formed plural air exhaust openings 23 in the side face 11b on the air exhaust side of the casing main body 11. These air exhaust openings 23 function as air exhaust portions for sending air away to the outside of the casing main body 11.

Inside the casing main body 11, a flow path communicating from the air intake opening 21 to the air exhaust opening 23 is formed. Upon driving the air cleaning apparatus 10, the air taken from the air intake opening 21 flows in the direction of an arrow F in FIG. 1, and is sent away from the air exhaust opening 23. Hereinafter, in the exemplary embodiment according to an aspect of the invention, the air intake side is referred to as an upstream side relative to the flow path, and the air exhaust side is referred to as a downstream side.

In the flow path of the casing main body 11, a photocatalyst filter 12 is arranged. The photocatalyst filter 12 of the exemplary embodiment has an approximately rectangular figure, has planes having an area approximately equal to the cross-section of the flow path and being parallel to each other, and is arranged so as to make the plane perpendicular to the air flow (arrow F) in the flow path. Meanwhile, in the exemplary embodiment, a photocatalyst filter 12a is arranged on the upstream side, and a photocatalyst filter 12b is arranged on the downstream side.

The photocatalyst filter 12 has a porous fiber layer such as nonwoven fabric, a deactivated titanium layer, and an active titanium layer on the deactivated titanium layer.

For the photocatalyst, mainly, titanium oxide (TiO₂) is used as a main body. In addition, zinc oxide (ZnO), cerium oxide (Ce₂O₃), terbium oxide (Tb₂O₃), magnesium oxide (MgO), erbium oxide (Er₂O₃), potassium tantalate (KTaO₃), cadmium sulfide (CdS), cadmium selenide (CdSe), and [Ru(bpy)₃]²⁺ and Co complexes can be applicable. Meanwhile, as the active titanium oxide, the use of fine particles of anatase crystal is desirable. As the fiber layer, the use of one, that has the basis weight of from 100 g/m² to 300 g/m², and, for pressure loss, an initial pressure loss of from 20 to 90 Pa at the standard wind velocity of 2.5 m/s, is preferable.

On the downstream side of the photocatalyst filter 12, an antibody filter 15 is provided. The antibody filter 15 may have the same dimension and shape as those of the photocatalyst filter 12.

The antibody filter 15 includes a harmful substance removal material composed of an antibody supported on a carrier.

The carrier may be formed of, for example, a humidity conditioning material. Examples of the humidity conditioning material include a fiber, which may constitute the carrier in the shape of woven fabric or nonwoven fabric. When the carrier is constituted by a fabric, in order to make the atmosphere surrounding the antibody have a humidity at which the antibody exhibits the activity, the fiber desirably contains a large amount of moisture.

The antibody is a protein that reacts specifically (antigen antibody-reaction) with a particular harmful substance (antigen), and has a molecular size of from 7 to 8 nm and a molecular figure of a Y letter. In the molecular figure of the Y letter of the antibody, a pair of branch parts are called Fab, and the backbone part is called Fc, and, among these, the Fab part catches a harmful substance.

The kind of the antibody corresponds to the kind of the harmful substance to be caught. Examples of harmful substances that are caught by the antibody include bacteria, fungi, viruses, allergens and mycoplasmas. Specifically, bacteria include, for example, gram-positive bacteria such as Staphylococcus (Staphylococcus aureus and Staphylococcus epidermidis), genus Micrococcus, anthrax bacillus, Bacillus cereus, hay bacillus and Propionibacterium acnes, and gram-negative bacteria such as Pseudomonas aeruginosa, Serratia marcescens, Burkholderia cepacia, pneumococcus, legionella bacteria and tubercle bacillus. Examples of fungi can include yeast, Aspergillus, Penicillius and Cladosporium. Examples of viruses can include influenza viruses, coronaviruses (SARS virus), adenoviruses and rhinoviruses. Examples of allergens can include pollen, mite allergens (decomposed material of mite), fungus spores and cat allergens (dandruff of a pet). Among these, bacteria and fungi are brought to bacteriostasis by a high adsorption effect, although they are not deactivated by the antibody. In contrast, viruses and allergens are disinfected or deactivated.

Regarding methods for producing the antibody, there can be mentioned, for example, a method of administering an antigen to an animal such as goat, horse, sheep and rabbit and purifying a polyclonal antibody from blood thereof; a method of performing cell fusion of a spleen cell of an animal having been administered with an antigen and a cultured cancer cell, and purifying a monoclonal antibody from body fluids (such as ascites) of an animal to which the culture fluid or the fused cell has been planted; a method of purifying an antibody from a culture fluid of genetically modified bacteria, plant cells or animal cells having been introduced with an antibody-producing gene; and a method of administering an antigen to a hen to allow it to lay an immune egg and purifying an egg antibody from egg yolk powder obtained by disinfecting and spray-drying the egg yolk fluid. Among these, the method of obtaining an antibody from an egg can easily give a large amount of antibodies to allow the cost reduction of the harmful substance removal material to be designed.

The carrier has desirably been subjected to antibacterial processing such as performing a coating containing an antibacterial agent and/or antifungal processing such as performing a coating containing an antifungal agent. Antibodies are protein basically, in particular, the egg antibody is food, and, further, protein other than the antibody may be accompanied, which constitute wonderful feed for the growth of bacteria and fungi. However, when the carrier has been subjected to antibacterial processing and/or antifungal processing, the growth of such bacteria and fungi are suppressed to make the long time storage possible. Examples of antibacterial/antifungal agents can include organic silicon quaternary ammonium salt-based ones, organic quaternary ammonium salt-based ones, biguanide-based ones, polyphenol-based ones, chitosan, silver-supported colloidal silica and zeolite-supported silver-based ones. For the processing method, there are a post-processing method in which an antibacterial/antifungal agent is impregnated into or coated onto a carrier made of a fiber, a raw yarn and raw cotton-modifying method in which an antibacterial/antifungal agent is kneaded at a synthesis step of the fiber that constitutes the carrier, and the like.

As the antibacterial agent and antifungal agent, an organic acid silver salt can be employed. Preferably, the organic acid silver salt has from 14 to 24 carbon atoms and is linear. The organic acid for constituting the silver salt is preferably a linear fatty acid. The fatty acid desirably has from 14 to 24 carbon atoms. When the number of carbon atoms is less than 14, the influence of steric hindrance is small, and the organic acid silver salt attacks the S—S bond of an antibody to generate destruction of the antibody. When the number of carbon atoms exceeds 24, on the other hand, the release amount of silver ions reduces due to the solubility product constant with silver, to decrease the antibacterial effect. For organic silver salts, there are descriptions in Research Disclosure, volumes 17029 and 29963. For the production method thereof, there is description, for example, in JP-A-2000-187298 (FUJIFILM Corporation). The constitution, in which the antibody filter according to the invention supports at least either one of an antibacterial agent and an antifungal agent, can decompose bad-smelling material by the photocatalyst, and, by combining the antibody filter, selectively deactivate bacteria and viruses. In particular, by using an antibody and an organic antibacterial agent in combination, it is possible to supply the antibody filter having an antibacterial effect, while maintaining the effect of selectively deactivating the antibody.

Regarding methods for fixing the antibody to the carrier, there can be mentioned a method of silanizing the carrier using γ-aminopropyltriethoxysilane or the like and, subsequently, introducing an aldehyde group to the carrier surface with glutaraldehyde to allow the aldehyde group and the antibody to form a covalent bond; a method of dipping an untreated carrier in an aqueous antibody solution to fix the antibody to the carrier thorough an ionic bond; a method of introducing an aldehyde group to a carrier having a specified functional group to allow the aldehyde group and the antibody to form a covalent bond; a method of allowing a carrier having a specified functional group to form an ionic bond with an antibody; and a method of coating a carrier with a polymer having a specified functional group and, subsequently, introducing an aldehyde group to allow the aldehyde group and the antibody to form a covalent bond. Here, as the specified functional group, there can be mentioned an NHR group (R is any alkyl group among methyl, ethyl, propyl and butyl, excluding H), an NH₂ group, an C₆H₅NH₂ group, an CHO group, a COOH group and a OH group.

In addition, there is also a method of converting a functional group on the carrier surface to another functional group using BMPA (N-β-Maleimidopropionic acid) or the like and, subsequently, allowing the functional group and an antibody to form a covalent bond (in the case of BMPA, a SH group is converted to a COOH group).

Furthermore, there is also a method of introducing a molecule (such as a Fc receptor and protein A/G) that is selectively bonded to the Fc part of an antibody to the carrier surface, to which the Fc of an antibody is bonded. On this occasion, the Fab that catches harmful substances exists outward relative to the carrier to lead to a high contact probability of harmful substances to the Fab, and, therefore, it is possible to effectively catch harmful substances.

Photocatalyst filters 12a, 12b and the antibody filter 15 are held by a filter cassette, and are arranged in prescribed positions by loading the filter cassette 50 to the casing main body 11. In the exemplary embodiment, the photocatalyst filters 12a, 12b and the antibody filter 15 have a shape of long plate, and are arranged so that the faces thereof are in the perpendicular direction to the flow path F to make the air flow possible, and, at the same time, make the shielding state possible when being seen with the naked eye.

In the flow path inside the casing main body 11, a light-emitting portion 14 for irradiating the photocatalyst filter 12 with light is provided. In the exemplary embodiment, the light-emitting portion 14 is arranged between the photocatalyst filter 12a on the upstream side and the photocatalyst filter 12b on the downstream side. The light-emitting portion 14 includes a light source that produces emission of UV rays of around from 300 nm to 420 nm, which is the wavelength to which the photocatalyst responds. In the exemplary embodiment, a fluorescent lamp is used as the light source of the light-emitting portion 14, but the source is not limited to it, and, for example, an LED (Light Emitting Diode) and other UV ray irradiation apparatuses may be used. Near the light-emitting portion 14 in the exemplary embodiment, a glow lamp for lighting the fluorescent lamp may be provided.

As shown in FIGS. 1 and 4, an air blast portion 16 is provided at a portion on the downstream side of the flow path of the casing main body 11 and just before the air exhaust opening 23 (near a portion on the upstream side). In the exemplary embodiment, an axial fan is used as the air blast portion 16. When it is driven, by the rotation of the fan, the air blast portion 16 sends the air inside the flow path away from the air exhaust opening 23 on the downstream side, and thus, in the flow path, such flow of air generates that the air is taken in from the air intake opening 21 on the upstream side to send the air along the flow path and send it away from the air exhaust opening 23 on the downstream side, as shown by the arrow F in FIG. 1. For the air blast portion 16, a sirocco fan and the like may be used, in place of the axial fan. Meanwhile, although the exemplary embodiment has such constitution that the air blast portion 16 is provided on the downstream side of the flow path, such constitution that the air blast portion 16 is provided on the upstream side of the flow path may be allowable, or such constitution that the air blast portions 16 are provided on both upstream and downstream sides of the flow path may be allowable.

For the air cleaning apparatus 10, further, inside the casing main body 11, there are provided a power supply circuit 32 for supplying power to the light-emitting portion 14 and the air blast portion 16, a motor-controlling portion 33, and a transformer 34 capable of transforming the voltage of the light-emitting portion 14. On the side face 11b on the air exhaust side of the casing main body 11, a power switch 24 is provided. Furthermore, on the air intake side 11a of the casing main body 11, an air volume-adjusting portion 26 that allows a user to adjust the flow volume of the air sent from the air blast portion 16 is provided.

In addition, as shown in FIG. 4, at a portion on the upstream side of the flow path and down stream side of the air intake opening 21, an after-mentioned light-shielding member 42 is provided for the purpose of preventing the leakage of light from the light-emitting portion 14 from the air intake opening 21 to the outside of the casing main body 11. This can prevent the irradiation of such light as UV rays, which are harmful to the human body, to the outside during the driving and ensure safety.

As shown in FIGS. 1 and 4, the air cleaning apparatus 10 of the exemplary embodiment is provided with the light-shielding member 44 between the light-emitting portion 14 and the antibody filter 15. In addition, in the flow path, further another light-shielding member 42 is provided near the downstream side of the air intake opening 21. Meanwhile, the constitution of the light-shielding members 42, 44 will be described later.

Next, the controlling system of the air cleaning apparatus 10 of the exemplary embodiment will be described.

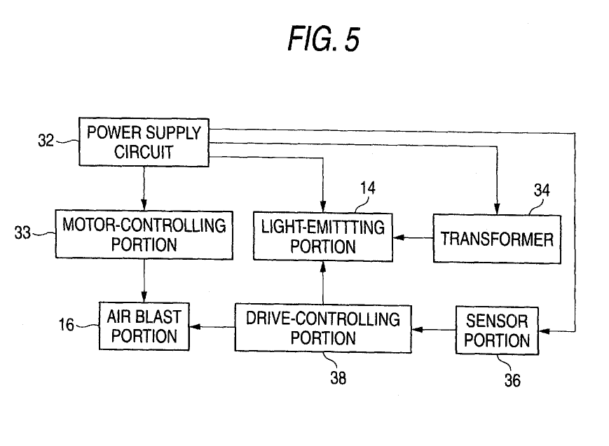

FIG. 5 is a block diagram showing the control system of the air cleaning apparatus of the exemplary embodiment. Meanwhile, in the exemplary embodiment as described below, for members that have constitution/function equivalent to members having been described, by giving the same or corresponding symbols in the drawings, description thereof will be simplified or omitted. At the driving of the air cleaning apparatus 10, by initiating the power supply circuit 32, a prescribed voltage is supplied to a motor-controlling portion 33, the light-emitting portion 14 and the transformer 34. By setting the transformer 34 to a prescribed frequency (for example, frequencies of 50 Hz and 60 Hz), the voltage relating to the driving of the light-emitting portion 14 can be switched. By driving the motor-controlling portion 33, the air blast portion 16 is driven and air begins to flow along the flow path of the casing main body 11. By initiating the driving of the light-emitting portion 14 at the same time as initiating the driving of the air blast portion 16, or in the vicinity of initiating the driving, the irradiation of light is initiated to generate active oxygen at the photocatalyst filter 12, and, at the same time, the active oxygen is diffused into the ambient atmosphere of the air cleaning apparatus 10 by the air flown by the air blast portion 16.

Here, the air cleaning apparatus 10 is provided with a sensor portion 36 for detecting the amount of organic materials in the atmosphere, and a drive-controlling portion 38 that is connected in such state that allows signals to be input and output to at least either one of the light-emitting portion 14 and the air blast portion 16. When the sensor portion 36 detects an organic material, it outputs the detection signal to the drive-controlling portion 38. The drive-controlling portion 38 can control at least either one of the light-emitting portion 14 and the air blast portion 16 on the basis of the detection signal related to the organic material. In the case of controlling the light-emitting portion 14, it can control the irradiation amount of light and the irradiation time of light. In addition, it may be provided with such function as setting the lighting of the light-emitting portion 14 to be driven intermittently, or a timer for terminating the irradiation. In the case of controlling the air blast portion 16, the amount of air to be sent and the time for sending air can be controlled. In addition, it may be provided with such function as setting the air blast portion 16 to be driven intermittently, or a timer for terminating the air blasting.

For odors to be detected by the sensor portion 36, for example, there are body odors, breath odors and alcoholic materials from human bodies, organic materials generated from feces and urine of pet animals, and the like. The sensor portion can also detect, for example, house dust such as ticks, dust and pollen, in addition to odors.

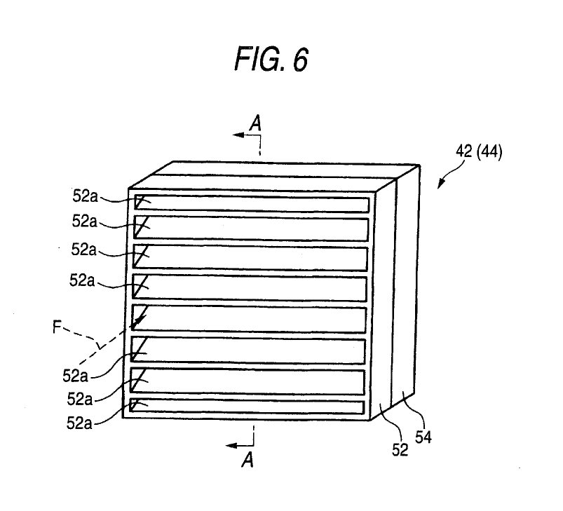

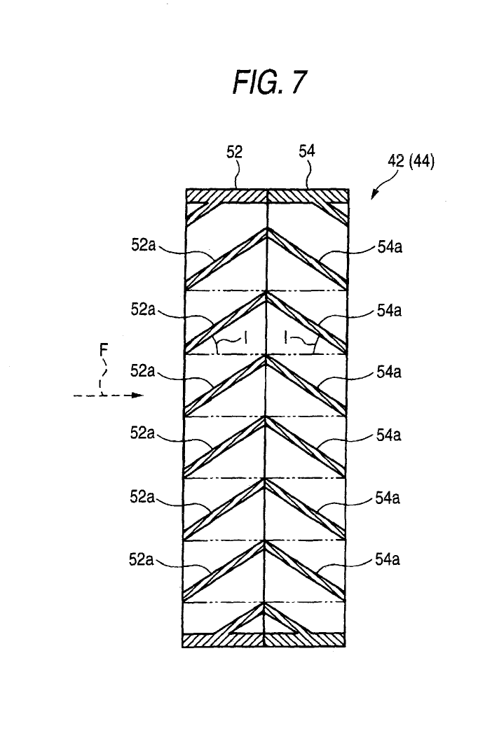

FIG. 6 is a perspective view showing the constitution of the light-shielding member. FIG. 7 is a cross-sectional view seen from the direction of the A-A line in FIG. 6. Each of the light-shielding members 42, has frame bodies 52, 54 in an approximately rectangular shape when being seen from the inflow direction of air (the arrow F in FIG. 6), and plural light-shielding plates 52a, 54a that are formed for the frame bodies 52, 54 and shield the transit of light irradiated from the light-emitting portion 14. In the exemplary embodiment, each of plural frame bodies 52, 54 having the same constitution, respectively, is stacked to be used as one light-shielding member 42 or one light-shielding member 44. The plural light-shielding plates 52a, 54a are arranged parallel to each other at an approximately equal intervals, wherein the spaces between each of the light-shielding plates 52a, 54a are communicated with each other from the upstream side to the downstream side, to allow the air flowing in from the face on the upstream side of the frame body 52 to pass through the face on the downstream side. All the plural light-shielding plates 52a, 54a have the same inclination angle I, which is preferably in the range of from 30 degrees to 50 degrees relative to the horizontal direction (right to left direction in FIG. 7) of the casing main body 11.

The light-shielding members 42, 44 in the exemplary embodiment are arranged in such state that plural frame bodies 52, 54 are stacked, respectively, and disposed, wherein the inclination directions of the light-shielding plates of adjacent frame bodies are inverse to each other. Meanwhile, the light-shielding members 42, 44 may be constituted of either one of the frame bodies 52, 54 and plural light-shielding plates 52a or 54a formed thereto.

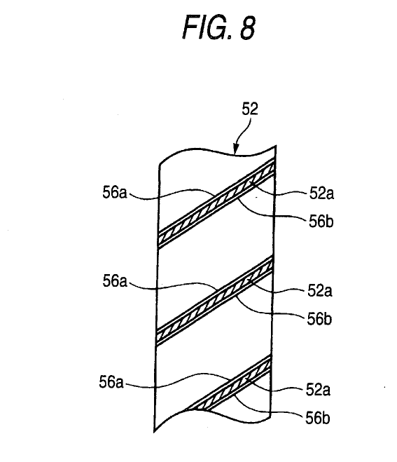

FIG. 8 is a partial cross-sectional view showing a modified example of the light-shielding member of the exemplary embodiment. As shown in FIG. 8, photocatalyst layers 56a, 56b may be formed for the upper face and the lower face of the light-shielding plate 52a. The photocatalyst layers 56a, 56b may have the same constitution as that of the photocatalyst filters 12a, 12b. For example, they may be constituted by sticking the photocatalyst filter to the upper and lower faces of the light-shielding plate 52a. The photocatalyst layers 56a, 56b may be formed for only either one of the upper and lower faces of the light-shielding plate 52a. This can prevent the irradiation of light to the downstream side with the help from the light-shielding plate 52a, due to the irradiation of light from the light-emitting portion 14 to the light-shielding plate 52a, and, at the same time, a photocatalyst reaction can be initiated at the photocatalyst layers 56a, 56b of the light-shielding plate 52a.

### EMBODIMENT

### Example

Next, on the basis of the air cleaning apparatus of the exemplary embodiment, tests for measuring samples in Examples and Comparative Examples were performed as described below. Meanwhile, it is assumed that air cleaning apparatuses for use in Examples and Comparative Examples have the same constitution as the above-described air cleaning apparatus unless otherwise designated, and the description will be omitted or simplified.

Antibody filters for use in Examples and Comparative Examples were prepared according to the following procedure.

(Nonwoven Fabric N-1)

A solution (25% by mass) of cellulose acetate (manufactured by ALDRICH Corp., total substitution degree: 2.4, number average molecular weight: 30,000) in acetone:water (97:3) was heated to 60° C., which was ejected along with air from a nozzle having a diameter of 0.1 mm at a spinning rate of 500 m/m to form nonwoven fabric. Thus nonwoven fabric N-1 having a thickness of 85 μm was obtained. A spinning cylinder was heated with a heater to 100° C. The average fiber diameter was measured with a SEM to give 8 μm. (In this specification, mass ratio is equal to weight ratio.)

(Fixation of Antibody)

An influenza virus antibody (IgY antibody) prepared by purifying an immune egg laid by a hen having been administered with an antigen was dissolved in a phosphate buffered saline so as to give an antibody concentration of 100 ppm. In the prepared liquid, a sample of the above-described nonwoven fabric N-1 was dipped at room temperature for 16 to 24 hours to provide the fabric surface with the antibody. The obtained sample was left at rest under such circumstances as 25° C. and 20% RH for 24 hours, and then was left at rest under such circumstances as 25° C. and 90% RH for 24 hours. Each of these operations was alternately repeated three times.

Next, photocatalyst filters in Examples and Comparative Examples for use in the measurement were prepared by the following procedure.

A photocatalyst coating agent (TKC-304, manufactured by TAYCA CORPORATION) was supported on a nonwoven fabric that was made of a polyester/acryl-based fiber with a diameter of 20 μm and had a thickness of 7 mm so as to give 7.5 g per 1 m², which was then dried at 100° C. for 3 minutes to prepare a photocatalyst filter.

(Arrangement of Filter to Air Cleaning Apparatus and Evaluation)

A frame was prepared for the photocatalyst and antibody filters. Then, in the constitution of the embodiment, in which a filter-holding portion capable of detachably holding the filter was provided, the antibody nano-filter N-1 prepared as described above was arranged at the lowermost stream of the air flow, a pair of photocatalyst filters were arranged on the upstream side, and a cold cathode tube that effectively emitted near UV rays was arranged therebetween. As the air blast portion, three axial fans were arranged at the lowermost stream.

### Example 1

An air cleaning apparatus, which was arranged with the antibody filter N-1 and a pair of photocatalyst filters, and the light-shielding member consisting of two frame bodies each having light-shielding plates (made of ABS resin having been subjected to an anti-UV treatment) with an inclination of 30 degrees inserted between the antibody filter and the photocatalyst filter on the downstream side, was used. The two frame bodies were arranged so as to have respective inclination directions of the light-shielding plates inverse to each other (refer to FIG. 7).

### Example 2

An air cleaning apparatus, which was arranged with the antibody filter N-1 and a pair of photocatalyst filters, and the light-shielding member consisting of one frame body having light-shielding plates (made of ABS resin having been subjected to an anti-UV treatment) with an inclination of 30 degrees inserted between the antibody filter and the photocatalyst filter on the downstream side, was used.

### Example 3

An air cleaning apparatus, which was arranged with the antibody filter N-1 and a pair of photocatalyst filters, and the light-shielding member consisting of two frame bodies each having light-shielding plates (made of ABS resin having been subjected to an anti-UV treatment) with an inclination of 45 degrees inserted between the antibody filter and the photocatalyst filter on the downstream side, was used. The two frame bodies were arranged so as to have respective inclination directions of the light-shielding plates inverse to each other (refer to FIG. 7).

### Example 4

An air cleaning apparatus, which was arranged with the antibody filter N-1 and a pair of photocatalyst filters, and the light-shielding member consisting of one frame body having light-shielding plates (made of ABS resin having been subjected to an anti-UV treatment) with an inclination of 45 degrees inserted between the antibody filter and the photocatalyst filter on the downstream side, was used.

### Example 5

An air cleaning apparatus, which was arranged with the antibody filter N-1 and a pair of photocatalyst filters, and the light-shielding member consisting of two frame bodies each having light-shielding plates (made of ABS resin having been subjected to an anti-UV treatment) with an inclination of 50 degrees inserted between the antibody filter and the photocatalyst filter on the downstream side, was used. The two frame bodies were arranged so as to have respective inclination directions of the light-shielding plates inverse to each other (refer to FIG. 7).

### Example 6

An air cleaning apparatus, which was arranged with the antibody filter N-1 and a pair of photocatalyst filters, and the light-shielding member consisting of one frame body having light-shielding plates (made of ABS resin having been subjected to an anti-UV treatment) with an inclination of 50 degrees inserted between the antibody filter and the photocatalyst filter on the downstream side, was used.

### Comparative Example 1

An air cleaning apparatus, which was arranged with the antibody filter N-1 and a pair of photocatalyst filters, and the light-shielding member consisting of two frame bodies each having light-shielding plates (made of ABS resin having been subjected to an anti-UV treatment) with an inclination of 25 degrees inserted between the antibody filter and the photocatalyst filter on the downstream side, was used. The two frame bodies were arranged so as to have respective inclination directions of the light-shielding plates inverse to each other (refer to FIG. 7).

### Comparative Example 2

An air cleaning apparatus, which was arranged with the antibody filter N-1 and a pair of photocatalyst filters, and the light-shielding member consisting of one frame body having light-shielding plates (made of ABS resin having been subjected to an anti-UV treatment) with an inclination of 25 degrees inserted between the antibody filter and the photocatalyst filter on the downstream side, was used.

### Comparative Example 3

An air cleaning apparatus, which was arranged with the antibody filter N-1 and a pair of photocatalyst filters, and the light-shielding member consisting of two frame bodies each having light-shielding plates (made of ABS resin having been subjected to an anti-UV treatment) with an inclination of 55 degrees inserted between the antibody filter and the photocatalyst filter on the downstream side, was used. The two frame bodies were arranged so as to have respective inclination directions of the light-shielding plates inverse to each other (refer to FIG. 7).

### Comparative Example 4

An air cleaning apparatus, which was arranged with the antibody filter N-1 and a pair of photocatalyst filters, and the light-shielding member consisting of one frame body having light-shielding plates (made of ABS resin having been subjected to an anti-UV treatment) with an inclination of 55 degrees inserted between the antibody filter and the photocatalyst filter on the downstream side, was used.

### Comparative Example 5

An air cleaning apparatus, in which an antibody filter N-1 and a pair of photocatalyst filters were arranged, was used.

(Evaluation of Odor Neutralization Effect)

The odor neutralization effect of air cleaning apparatuses was evaluated on the basis of ammonia concentration. After adjusting the initial ammonia (NH₃) concentration in a closed space (0.2 m³) in which the test was performed to 10 ppm, the air cleaning apparatus was driven and the ammonia concentration was measured with a detector tube after 15 minutes.

(Evaluation of Air Volume)

The air volume was obtained as follows. A tube having a size of height 26 cm, breadth 7 cm and length 30 cm was prepared and attached to the blowout opening. Then, wind velocity (m/s) was measured at ten points, values of which were averaged to give the air volume (m³/min).

(Evaluation of UV Intensity at Antibody Filter)

It was measured with a UV power meter (C9536-01/H9958) manufactured by Hamamatsu Photonics K.K.

(Evaluation of Efficiency for Deactivating Viruses)

Air cleaning apparatuses of the aforementioned conditions were operated under the same circumstances for two weeks. Then, the evaluation of deactivating viruses was performed for respective antibody filters.

As a virus liquid for test, a purified influenza virus was used after ten times dilution with PBS. Each of aforementioned samples was cut in the shape of 5 cm square, which was attached and fixed to the center of a virus spray test apparatus. The virus liquid for test was charged in a nebulizer provided on the upstream side, and an apparatus for collecting viruses was attached on the downstream side. Compressed air was sent from an air compressor to spray the virus for test from a spray opening of the nebulizer. On the downstream side of a mask, a gelatin filter was provided, and, while absorbing the air in the test apparatus at an absorption flow volume of 10 L/min for 5 minutes, passing virus mist was collected.

After the test, the gelatin filter having caught the virus was collected, and, by a TCID50 method (median tissue culture infectious dose method) using MDCK cells, the viral infectivity titer after the passage of the sample was obtained. From the comparison of the viral infectivity titer of gelatin filters in presence or absence of the sample, the removal ratio of viruses in one passage was calculated for respective samples. Results are shown in Table 1 below.

TABLE 1

| | Light-shielding member | | | NH₃ | Removal |
|---|---|---|---|---|---|
| | (The number of frame body; | UV | Air | concentration | ratio of |
| | The inclination angle of the | intensity | volume | after 15 min | viruses for |
| | light-shielding plates) | (μW/cm²) | (m^{3/}min) | (ppm) | one passage |
| Example 1 | 2; | 0 | 0.5 | 0.5 | 87 |
| | inclination 30° | | | | |
| Example 2 | 1; | 12 | 0.5 | 0.5 | 87 |
| | inclination 30° | | | | |
| Example 3 | 2; | 0 | 0.5 | 0.5 | 88 |
| | inclination 45° | | | | |
| Example 4 | 1; | 10 | 0.5 | 0.5 | 87 |
| | inclination 45° | | | | |
| Example 5 | 2; | 0 | 0.5 | 0.5 | 87 |
| | inclination 50° | | | | |
| Example 6 | 1; | 10 | 0.5 | 0.5 | 88 |
| | inclination 50° | | | | |
| Comparative | 2; | 60 | 0.5 | 0.5 | 55 |
| Example 1 | inclination 25° | | | | |
| Comparative | 1; | 80 | 0.5 | 0.5 | 50 |
| Example 2 | inclination 25° | | | | |
| Comparative | 2; | 0 | 0.25 | 2 | 60 |
| Example 3 | inclination 55° | | | | |
| Comparative | 1; | 20 | 0.35 | 1 | 64 |
| Example 4 | inclination 55° | | | | |
| Comparative | None | 100 | 0.5 | 0.5 | 40 |
| Example 5 | | | | | |

As shown for Examples 1-5, it is understood that, by setting the inclination angle of the light-shielding plates in the range of from 30 degrees to 50 degrees, the UV intensity at the antibody filter could be suppressed to 10 μW/cm² or less, the air volume of 0.5 m³/min could be assured and the removal ratio of viruses in one passage was so high as 87-88%.

For Comparative Examples 1 and 2, the UV intensity at the antibody filter was so high as 60 μW/cm² or more caused by setting the inclination angle of the light-shielding plates to 25 degrees, and, consequently, the filter effect of the antibody filter lowered to give such low removal ratio of viruses in one passage as 50-55%. For Comparative Examples 3 and 4, although the UV intensity was suppressed to 20 μW/cm² or less, the interference of the air flow occurred at the light-shielding plates to give such low air volume as 0.25-0.35 cm³/min. Further, the NH₃ concentration after 15 minutes was so high as 1 ppm or more also to show so low value of the removal ratio of viruses in one passage as 60-64%. For Comparative Example 5, caused by providing no light-shielding member, the antibody filter was largely affected by UV rays to reduce the removal ratio of viruses in one passage to 40%.

Next, under the same conditions as those for the above-described measurement test, measurements were performed for constitutions of Examples and Comparative Examples as described below.

### Example 7

In the same way as in Example 3, an air cleaning apparatus, which was arranged with the antibody filter N-1 and a pair of photocatalyst filters, and two louvers (made of ABS resin having been subjected to an anti-UV treatment) each having light-shielding plates with an inclination of 45 degrees inserted between the antibody filter and the photocatalyst filter on the downstream side, was used. The two louvers were arranged so as to have respective inclination directions of the light-shielding plates inverse to each other (refer to FIG. 7).

### Example 8

An air cleaning apparatus, which was arranged with the antibody filter N-1 and a pair of photocatalyst filters, two louvers (made of ABS resin having been subjected to an anti-UV treatment) each having light-shielding plates with an inclination of 45 degrees inserted between the antibody filter and the photocatalyst filter on the downstream side, and TKC304 1 g/m² coated on the upper surface of the light-shielding plates (to which UV light was irradiated) as a photocatalyst layer, was used. The two louvers were arranged so as to have respective inclination directions of the light-shielding plates inverse to each other (refer to FIG. 7).

### Comparative Example 6

An air cleaning apparatus, which was arranged with the antibody filter N-1 and a pair of photocatalyst filters, was used. It had such constitution as being not arranged with the light-shielding member.

### Comparative Example 7

An air cleaning apparatus, which was arranged with the antibody filter N-1 and a pair of photocatalyst filters, and a baffle plate having a surface perpendicular to the flow path inserted between the antibody filter and the photocatalyst filter on the downstream side, was used. Results of measurements are shown in Table 2 below.

TABLE 2

| | | | | NH₃ | |
|---|---|---|---|---|---|
| | | UV | Air | concentration | Removal ratio of |
| | Light-shielding | intensity | volume | after 15 min | viruses for |
| | member | (μW/cm²) | (m^{3/}min) | (ppm) | one passage |
| Example 7 | 2 louvers each having | 0 | 0.5 | 0.5 | 88 |
| | light-shielding plates with | | | | |
| | an inclination of 45° | | | | |
| Example 8 | 2 louvers each having | 0 | 0.5 | 0 | 88 |
| | light-shielding plates with | | | | |
| | an inclination of 45° | | | | |
| Comparative | None | 100 | 0.5 | 0.5 | 40 |
| Example 6 | | | | | |
| Comparative | Baffle plate | 0 | 0.2 | 2 | 50 |
| Example 7 | | | | | |

For Example 7, it was understood that, by providing two louvers, the UV intensity at the antibody filter could be suppressed to 0 μW/cm², the air volume of 0.5 m³/min could be assured and the removal ratio of viruses in one passage showed such a high value as 88%. For Example 8, by forming the photocatalyst layer on the surfaces of the light-shielding plates of the two louvers, NH₃ after 15 minutes was not detected to give substantially 0 ppm. For Comparative Example 6, as the result of not providing the light-shielding member, the antibody filter was largely influenced by UV rays to reduce the removal ratio of viruses in one passage to 40%. When the baffle plate was provided as in Comparative Example 7, although UV intensity at the antibody filter could be suppressed, the air volume lowered and the concentration of NH₃ after 15 minutes was so high as 2 ppm, and the removal ratio of viruses in one passage reduced to 50%.

Next, for an air cleaning apparatus having an antibody filter including an organic acid silver salt, the effect of deactivating viruses and the antibacterial activity were measured using Examples and Comparative Examples as described below.

(Preparation of Carrier Nonwoven Fabric)

A solution (25% by mass) of cellulose acetate (manufactured by ALDRICH Corp., total substitution degree: 2.4, number average molecular weight: 30,000) in acetone:water (97:3) was heated to 60° C., which was ejected along with air from a nozzle having a diameter of 0.1 mm at a spinning rate of 500 m/m to form nonwoven fabric. Thus carrier nonwoven fabric N-1 having a thickness of 4 mm was obtained. A spinning cylinder was heated with a heater to 100° C. The average fiber diameter was measured with a SEM to give 8 μm.

(Preparation of Coating Liquid)

The preparation of a coating liquid 1 will be described. An egg yolk liquid of an immune egg laid by a hen having been administered with an antigen was spray-dried to give dried egg yolk powder. Subsequently, the dried egg yolk powder was defatted with ethanol, which component was removed. Then, resulting product was dried under a reduced pressure to give defatted egg yolk powder as an antibody material. The defatted egg yolk powder was purified, for which the purity of an influenza virus antibody (IgY antibody) was measured to give 3% by mass. Subsequently, the defatted egg yolk powder was suspended in purified water so as to give an antibody concentration of 100 ppm. The liquid was referred to as the coating liquid 1.

The preparation of a coating liquid 2 will be described. To the coating liquid 1, a silver behenate (carbon atoms: 22) suspension was mixed and adjusted to give a silver behenate concentration of 200 ppm. The obtained liquid was referred to as the coating liquid 2.

The preparation of a coating liquid 3 will be described. To the coating liquid 1, a silver laurate (carbon atoms: 12) suspension was mixed and adjusted to give a silver laurate concentration of 118 ppm (to match with silver behenate in molar number). The obtained liquid was referred to as the coating liquid 3.

The preparation of a coating liquid 4 will be described. To the coating liquid 1, a silver myristate (carbon atoms: 14) suspension was mixed and adjusted to give a silver myristate concentration of 134 ppm (to match with silver behenate in molar number). The obtained liquid was referred to as the coating liquid 4.

The preparation of a coating liquid 5 will be described. To the coating liquid 1, a silver cerotate (carbon atoms: 26) suspension was mixed and adjusted to give a silver cerotate concentration of 230 ppm (to match with silver behenate in molar number). The obtained liquid was referred to as the coating liquid 5.

The preparation of a coating liquid 6 will be described. A silver behenate (carbon atoms: 22) suspension adjusted to give a silver behenate concentration of 200 ppm was referred to as the coating liquid 6.

(Preparation of Filter)

In the coating liquid 1, the aforementioned carrier nonwoven fabric N-1 was dipped at room temperature for 5 minutes to provide the carrier surface with the antibody. The obtained sample was compressed with a roller having a face pressure of 10 MPa, and the moisture content thereof was measured to give 500%. Further, when the sample was dried under such atmosphere as 50° C. and 30% RH so as to give a moisture content of 1% or less, the moisture content reached 1% after 3 hours. The thus obtained sample was referred to as an antibody filter F1 of Comparative Example 8.

Antibody filters F2-F6 were prepared in the same way as the antibody filter F1, except for replacing the coating liquid 1 with coating liquids 2-6, respectively. Thus, antibody filters F2-F5, which were provided with the antibody and organic acid silver salt on the carrier surface, were prepared. Then, the antibody filter F2 including the coating liquid 2 was called the sample of Example 9, the antibody filter F3 including the coating, liquid 3 was called the sample of Comparative Example 9, the antibody filter F4 including the coating liquid 4 was called the sample of Example 10, and the antibody filter F5 including the coating liquid 5 was called the sample of Comparative Example 10. Two types of filters, the antibody filter F1 and the antibody filter F6 including the coating liquid 6 were stacked and disposed to be called the sample of Comparative Example 11.

### (Evaluation of Efficiency for Deactivating Viruses)

For antibody filters F1 to F5 in Comparative Examples and Examples, the efficiency for deactivating viruses was evaluated just after the preparation of samples.

For the virus liquid for test, a liquid obtained by ten times diluting a purified influenza virus with PBS (virus concentration: 200,000 plaque/mL) was used. Each of the samples was cut in the shape of 5 cm square, which was attached and fixed to the center of the virus spray test apparatus. The virus liquid for test was charged in a nebulizer provided on the upstream side, and an apparatus for collecting viruses was attached on the downstream side. Compressed air was sent from an air compressor to spray the virus for test from a spray opening of the nebulizer. On the downstream side of the mask, a gelatin filter was provided, and, while absorbing the air in the test apparatus at an absorption flow volume of 10 L/min for 5 minutes, passing virus mist was collected.

After the test, the gelatin filter having caught the virus was collected, and, by a TCID50 method (median tissue culture infectious dose method) using MDCK cells, the viral infectivity titer after the passage of the sample was obtained. From the comparison of the viral infectivity titer of gelatin filters in presence or absence of the sample, the removal ratio of viruses in one passage was calculated for respective samples. Results are shown in Table 3.

(Evaluation of Antibacterial Activity)

For antibody filters F1 to F5 in Comparative Examples and Examples, an antibacterial activity test was performed just after the preparation of samples. The test method according to JIZ2801: 2000 was employed.

For bacteria to be tested, Staphylococcus aureus subsp. aureus NBRC 12732 (Staphylococcus aureus) pre-cultured with a standard agar medium were used. Such cultured bacteria were dispersed and diluted with 1/500 nutrient broth to prepare a test bacteria liquid. The test bacteria liquid 0.4 mL was inoculated to respective filters placed in a sterilized petri dish, which were cultivated at 35° C. for 24 hours. After the cultivation, bacteria were washed out from respective test fabrics with 10 mL of Soybean Casein digest Broth containing lecithin/polysorbate 80 to measure the number of bacteria in respective test fabrics by the agar plate cultivation method. Further, the number of bacteria just after the inoculation was also measured to give 1.8×10⁵. Results are shown in Table 3.

TABLE 3

| | | Carbon | Effect for | | Anti bacterial |
|---|---|---|---|---|---|
| | | number of | deactivating | | activity |
| | | organic | viruses | Number | (Number of |
| | Antibody | acid | (removal ratio | of | bacteria |
| | filter | silver | in one passage) | filters | after test) |
| Comparative | F1 | — | 93% | 1 | 1.5 × 10³ |
| Example 8 | | | | | |
| Example 9 | F2 | 22 | 93% | 1 | Not detected |
| Comparative | F3 | 12 | 25% | 1 | Not detected |
| Example 9 | | | | | |
| Example 10 | F4 | 14 | 75% | 1 | Not detected |
| Comparative | F5 | 26 | 93% | 1 | 1.5 × 10³ |
| Example 10 | | | | | |
| Comparative | F1 and F6 | 22 | 93% | 2 | Not detected |
| Example 11 | | | | | |

As is clear from Table 3, antibody filters obtained according to the production method of the invention has a high virus removal ratio just after the production of the sample, and can maintain the virus-deactivating capability after storage. When the carbon number was 26, the antibacterial effect was lost. It was considered that an increased solubility product constant lead to the suppression of the slow release of silver ions. When the carbon number was 12, it was considered that the destruction of antibodies by silver ions occurred to result in the lowering of deactivation efficiency by antibodies. In addition, the effect having normally been expressed with two filters, that is, the antibacterial filter and the antibody filter could be realized with one filter.

Next, for the case where each of the above-described carrier filters were arranged to the air cleaning apparatus, the odor neutralization effect was evaluated.

(Preparation of Photocatalyst Filter)

The photocatalyst coating agent (TKC-304, manufactured by TAYCA CORPORATION) was supported on a nonwoven fabric that was made of a polyester-based fiber with a diameter of 20 μm and had a thickness of 8 mm and a basis weight of 170 g/m² so as to give 20 g per 1 m², which was dried at 120° C. for 3 minutes to prepare a photocatalyst filter.

(Preparation of Activated Carbon Filter)

For the purpose of providing a filter with an odor neutralization effect in a short time by adsorption, while using a nonwoven fabric having a reduced basis weight and low pressure loss, an activated carbon nonwoven fabric filter was prepared. On a nonwoven fabric that was made of polyester/vinylon-based fiber with a diameter of from 30 to 50 μm and had a thickness of 0.5 mm and a basis weight of 50 g/m², acrylic resin was coated, on which an activated carbon (KURARAYCOAL GG) 40 g/m² was supported to prepare an activated carbon filter. The antibody nano-filter F2 prepared as above was arranged at the lowermost stream of the air flow in the constitution of the embodiment, in which a frame was made for the photocatalyst and antibody filter and a filter-holding portion capable of detachably holding the filter was provided, a pair of photocatalyst filters on the upstream side, and a cold cathode tube that effectively emitted near UV rays were arranged therebetween. The activated carbon filter was stacked and arranged on the surface inverse to the photocatalyst surface facing to the cold cathode tube in order to allow UV light to effectively irradiate the titanium surface. As the air blast portion, three axial fans were arranged at the lowermost stream.

In the filter constitution 1 in Comparative Example 12, the antibody filter F2 and a pair of filters each consisting of a set formed by stacking the photocatalyst filter and the activated carbon filter were arranged to an air cleaning apparatus.

In the filter constitution 2 in Example 11, the antibody filter F2 and a pair of filters each consisting of a set formed by stacking the photocatalyst filter and the activated carbon filter were arranged to an air cleaning apparatus, and two louvers (made of ABS resin having been subjected to an anti-UV treatment) each having light-shielding plates with an inclination of 45 degrees were inserted between the antibody filter and the photocatalyst filter on the downstream side.

In the filter constitution 3 in Example 12, the activated carbon nonwoven fabric was stacked to the antibody filter F2 and a pair of filters each consisting of a set formed by stacking the photocatalyst filter and the activated carbon filter, which was arranged to an air cleaning apparatus. The activated carbon nonwoven fabric was stacked and arranged on the surface inverse to the photocatalyst surface facing to the cold cathode tube in order to allow UV light to effectively irradiate the titanium surface.

In the filter constitution 4 in Example 13, one louver (made of ABS having been subjected to an anti-UV treatment) having light-shielding plates with the inclination of 45 degrees was inserted between the antibody filter and the photocatalyst filter on the downstream side in the air cleaning apparatus of Example 12.

In the filter constitution 5 in Example 14, two louvers (made of ABS having been subjected to an anti-UV treatment) each having light-shielding plates with the inclination of 45 degrees were inserted between the antibody filter and the photocatalyst filter on the downstream side in the air cleaning apparatus of Example 12.

### (Evaluation of Odor Neutralization Effect)

The odor neutralization effect of air cleaning apparatuses was evaluated on the basis of ammonia (NH₃) concentration. After adjusting the initial ammonia (NH₃) concentration in a closed space (1 m³) in which the test was performed to 10 ppm, the air cleaning apparatus was driven and the ammonia concentration was measured with a detector tube after 15 minutes.

(Evaluation of Air Volume)

The air volume was obtained as follows. A tube having a size of height 26 cm, breadth 7 cm and length 30 cm was prepared and attached to the blowout opening. Then, wind velocity (m/s) was measured at ten points, values of which were averaged to give the air volume (m³/min).

(Evaluation of UV Intensity at Antibody Filter)

It was measured with a UV power meter (C9536-01/H9958) manufactured by Hamamatsu Photonics K.K.

(Evaluation of Efficiency for Deactivating Viruses)

Air cleaning apparatuses of the aforementioned conditions were operated under the same circumstances for two weeks. Then, the evaluation of deactivating viruses of respective antibody filters was performed in the same way as described above. Results of the measurements are shown in Table 4 below.

TABLE 4

| | | | | NH₃ | Removal |
|---|---|---|---|---|---|
| | | | Air | concentration | ratio of |
| | Filter | UV intensity | volume | after | viruses in |
| | constitution | (μW/cm²) | (m³/min) | 15 min (ppm) | one passage |
| Comparative | Filter | 100 | 1 | 2 | 40 |
| Example 12 | constitution 1 | | | | |
| Example 11 | Filter | 0 | 1 | 2 | 90 |
| | constitution 2 | | | | |
| Example 12 | Filter | 10 | 0.9 | 1 | 86 |
| (activated | constitution 3 | | | | |
| carbon) | | | | | |
| Example 13 | Filter | 0 | 0.9 | 1 | 90 |
| (activated | constitution 4 | | | | |
| carbon + | | | | | |
| one louver) | | | | | |
| Example 14 | Filter | 0 | 0.9 | 1 | 90 |
| (activated | constitution 5 | | | | |
| carbon + | | | | | |
| two louvers) | | | | | |

It was understood that the carrier filter, which supported activated carbon on low pressure loss nonwoven fabric, had a UV-shielding effect. In addition, it was understood that an air cleaning apparatus provided with the carrier filter supporting activated carbon could increase the odor neutralization performance.

### INDUSTRIAL APPLICABILITY

According to the present invention, it is possible to provide an air cleaning apparatus that can prevent the reduction of the filter effect caused by the irradiation of UV rays to the antibody filter, and can prevent the lowering of the air volume.

The entire disclosure of each and every foreign patent application from which the benefit of foreign priority has been claimed in the present application is incorporated herein by reference, as if fully set forth.

1. An air cleaning apparatus for decomposing an organic material using a photocatalyst, the apparatus comprising:
a casing main body that includes:
an air intake portion that takes air in an inside of the casing main body; and
an air exhaust portion that sends air away to an outside of the casing main body;
an air blast portion that sends air to a flow path formed between the air intake portion and the air exhaust portion;
a photocatalyst filter that has a layer including a photocatalyst and is arranged in the flow path;
a light-emitting portion that irradiates the photocatalyst filter with light; and
an antibody filter that includes a harmful substance removal material constituted by supporting an antibody on a carrier and is arranged in the flow path, wherein:
a first light-shielding member that allows the air to flow and shields transit of the light in a state seen from the air flow direction is provided between the light-emitting portion and the antibody filter; and
the first light-shielding member includes:
at least one frame body that is arranged in the flow path; and
a plurality of light-shielding plates that are formed on the at least one frame body and arrayed in such a state as being inclined at the same angle respectively.

2. The air cleaning apparatus according to claim 1,
wherein at least either one of an antibacterial agent and an antifungal agent is supported on the antibody filter.

3. The air cleaning apparatus according to claim 2,
wherein the antibacterial agent and the antifungal agent is an organic acid silver salt.

4. The air cleaning apparatus according to claim 3,
wherein the organic acid silver salt has from 14 to 24 carbon atoms and is linear.

5. The air cleaning apparatus according to claim 1,
wherein the first light-shielding member includes a plurality of frame bodies each having the plurality of light-shielding plates, the plurality of frame bodies being disposed in a superimposed state, and
adjacent two frame bodies are arranged so as to have respective inclination directions of the plurality of light-shielding plates inverse to each other.

6. The air cleaning apparatus according to claim 1,
wherein each of the plurality of light-shielding plates is inclined in a range of from 30 degrees to 50 degrees relative to the horizontal direction.

7. The air cleaning apparatus according to claim 1, further comprising:
a second light-shielding member arranged near a lower stream side of the air intake portion in the flow path, the second light-shielding member being the same as the first light-shielding member.
